(19) United States
(12) Patent Application Publication
(10) Pub. No.: US 2023/0335301 A1
(43) Pub. Date: Oct. 19, 2023

(54) System and Method for the Structural Detection of Biological Systems and Their Axial Nodes

(71) Applicant: Nima Veiseh, Grand Haven, MI (US)

(72) Inventor: Nima Veiseh, Grand Haven, MI (US)

(21) Appl. No.: 18/301,882

(22) Filed: Apr. 17, 2023

Related U.S. Application Data
(60) Provisional application No. 63/331,657, filed on Apr. 15, 2022.

(51) Int. Cl. G16H 70/40; G16H 50/70

(52) U.S. Cl. CPC G16H 70/40; G16H 50/70

(57) ABSTRACT

Disclosed is a system and method of structurally detecting biological systems and their axial nodes.

The current application claims a priority to the U.S. provisional patent application Ser. No. 63/331,657 filed on Apr. 15, 2022. The current application is filed on Apr. 17, 2023 while Apr. 15, 2023 was on a weekend.

### FIELD OF THE INVENTION

The present invention relates generally to a method for detection, system and subsystem characterization. More specifically, the present invention is a method for characterization used in the endocannabinoid system and its space.

### BACKGROUND OF THE INVENTION

The present invention is a novel methodology for recursively reverse engineering a digital twin of a subsystem, the most typical use of this is biological subsystems and their responses, especially but not limited to the endocannabinoid system and its space.

### BRIEF DESCRIPTION OF THE DRAWINGS

FIG. 1 is a diagram of the present invention.

### DETAIL DESCRIPTIONS OF THE INVENTION

All illustrations of the drawings are for the purpose of describing selected versions of the present invention and are not intended to limit the scope of the present invention.

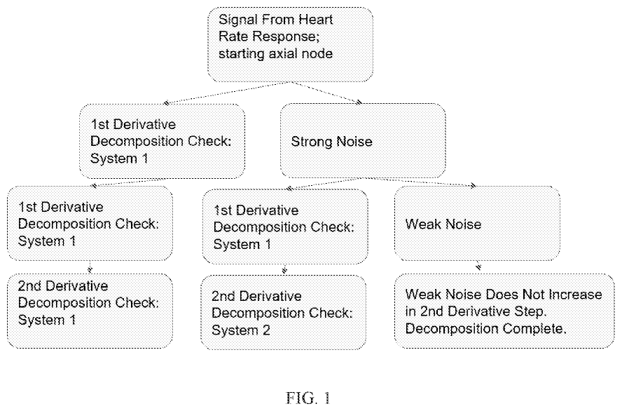

As can be seen in FIG. 1, the present invention is a novel methodology for recursively reverse engineering a digital twin of a subsystem, the most typical use of this is biological subsystems especially but not limited to the endocannabinoid system and its space. A large gap that this is filling is from the large amount of restriction with regards to direct clinical trial work in order to define specific pathways. However, this invention advances recent computational and distributed data infrastructure to perform in a novel way, this algorithmic clinical systemic characterization and evaluations using micro-level clinical data and observations to deduce the structure of biological systems and subsystems. For example, by monitoring the heartbeat, the present inventor can take the first derivative of a heartbeat as the system overall is working, and the second derivative can identify the health of the relationship between the autonomic system (by monitoring the exogenous system interactions) where are able to recursively compare the first and second derivative of system analysis of system components under incremental sensitivity testing until the present inventor has an understanding of core mechanisms. The characterization of its general structure helps employ all forms of research, drug delivery, discovery and development and non-invasively of critical life functions checking, because the present inventor is observing the weighted movement of the system not by its states but by its balance. This allows the present inventor to characterize biological systems with less invasive techniques than in the past, like when an animal may need to be killed in a lab study in order to perform the necessary tissue extraction to perform a system level characterization.

The axial node is the most minor component of a system that is both: a) mechanically independent, and b) emerges distinctly as a signal visibly decomposed from noise, error term, or interaction effects.

For example, the present inventor can start with an axial node like the “peripheral nervous system”. When the present inventor monitors the signals in that system, from a 1st derivative perspective, the present inventor can then see there are potentially more nodes. The present inventor then employs a stepwise breadth-first search to examine the scope of all potential nodes that may be candidates. After resolving the primary candidates to proceed with by using an identification mechanism that either: passes a measurement threshold, or a clinical assessment threshold, then the present inventor proceeds to decompose again. Thus, the present inventor then decomposes it again, into 2 systems, or Axial nodes, of “autonomic” and “somatic” systems. Looking at the balance of concurrent signals between these from the 1st derivative perspective, the present inventor may not see the signal indicating further decomposition. In order to confirm that they have reached an end node in this systemic decomposition, the present inventor then perform the 2nd derivative over the difference in the difference between the two systems (or n-number of permutations that the systems could be interacting) in order to exhaust the likelihood that other sub axial nodes exist that need to be teased out of the remaining noise data.

The precision and accuracy behind the methodology have an enhancement mechanism rooted in the inclusion and cyclical validation of these systems, clinical examinations, and subject matter expertise. The system representations can be communicated to clinical and research professionals for further discussion, augmentation, and validation.

Similarly, the Axial nodes and interaction effects go beyond the characterization of the mechanical system itself, and the axial nodes can be virtualized as “receptors”. This enables a contextual representation of the notes, that allows the present inventor to understand not just the discrete representation of the signal, but also the continuous and time decay functionality of it. For example, the present inventor has identified a phenomenon called the “Entourage accumulation effect” where receptions within the Endocannabinoid system (or psychedelic) produce different physiological and psychological effects based on a number of factors, including but not limited to, recency, chemical composition, cannabinoid composition, patient sensitivity, etc.

The prior art for the present invention includes the following:
1. U.S. Pat. No. 8,631,126B2
2. WO2006091528A3
3. WO2009110991A2
4. US20220013202A1
5. “Markets With Memory”, by Nima Veiseh, 2019. ISBN: 9781700165121
6. US20190362219A1
7. DK2047392T3
8. U.S. Pat. No. 7,027,621B1

Although the invention has been explained in relation to its preferred embodiment, it is to be understood that many other possible modifications and variations can be made without departing from the spirit and scope of the invention.

What is claimed is:

1. A method of structurally detecting biological systems and their axial nodes, the method comprising the steps of:
(A) executing an algorithmic methodology for decomposition of a known, partially-known, or yet-to-be-characterized biological system into its constituent parts;
(B) executing an entourage acclimation validation and decomposition, with the rate of decay estimates, to inform future product understanding, development, reformation, and gain of function;
(C) executing an algorithmic methodology for accelerating drug and API (Active Pharmaceutical Ingredient) validation and discovery; and
(D) leveraging clinical, patient, and research expertise, reported to, by, or self-reported, to characterize systems based on 1st, 2nd, and/or 3rd party data.
